(19) United States
(12) Patent Application Publication
(10) Pub. No.: US 2025/0223529 A1
(43) Pub. Date: Jul. 10, 2025

(54) OPTIMIZED LIGNOCELLULOSIC FEEDSTOCK DIGESTION

(71) Applicants: Lee Duane Hansen, Saratoga Springs, UT (US); Jaron Conly Hansen, Springville, UT (US); Zachary T. Aanderud, Orem, UT (US)

(72) Inventors: Lee Duane Hansen, Saratoga Springs, UT (US); Jaron Conly Hansen, Springville, UT (US); Zachary T. Aanderud, Orem, UT (US)

(21) Appl. No.: 18/984,303

(22) Filed: Dec. 17, 2024

Related U.S. Application Data
(60) Provisional application No. 63/619,487, filed on Jan. 10, 2024.
(60) Provisional application No. 63/655,684, filed on Jun. 4, 2024.

(51) Int. Cl. C12M 1/107; C12M 1/34; C12N 1/14

(52) U.S. Cl. CPC C12M 21/04; C12M 41/12; C12M 41/26; C12N 1/14

(57) ABSTRACT

The disclosed technologies provide a multi-stage system and process for converting lignocellulosic feedstock into high-quality methane-rich biogas. The technologies include an initial anaerobic secretome bioreactor (ASB1) operating under low-moisture, mesophilic conditions, which maximizes enzymatic hydrolysis, acetogenesis, and partial acidogenesis of the feedstock while producing biogas. ASB1 produces a semi-solid effluent rich in partially degraded organic compounds, residual lignin, and microbial biomass. A subsequent thermophilic anaerobic secretome bioreactor (ASB2) accepts this effluent as a substrate and facilitates further enzymatic breakdown and pasteurization through thermophilic microbial activity, producing an effluent optimized for methanogenesis. The final methanogenic digestion stage processes ASB2's effluent, converting it into methane with minimal carbon dioxide content, thus enhancing biogas quality and yield. This multi-stage process efficiently breaks down complex organic materials while minimizing inhibitory byproducts, enabling high-efficiency biogas production suitable for energy applications.

### RELATED APPLICATION(S)

This application claims priority to U.S. Provisional Application No. 63/619,487, filed on Jan. 10, 2024, and U.S. Provisional Application No. 63/655,684, filed on Jun. 4, 2024, with respect to subject matter disclosed in those applications. Each of the referenced provisional applications is incorporated herein by reference in its entirety.

This application includes additional subject matter not disclosed in the aforementioned provisional applications; therefore, the effective filing date for claims reciting this new subject matter is the filing date of the present utility application.

### BACKGROUND

Biogas production through anaerobic digestion of organic waste offers substantial potential for generating renewable energy in the form of methane. However, direct anaerobic digestion of lignocellulosic feedstocks, such as green waste and other fibrous plant materials, remains challenging. These feedstocks contain high levels of lignin, cellulose, and hemicellulose, which are resistant to breakdown under standard anaerobic conditions, resulting in low biogas yields. Conventional pretreatment methods, including thermal and chemical processes, are frequently employed to release digestible sugars from lignocellulose. However, these methods can be cost-prohibitive and energy-intensive due to high temperature and chemical requirements, which limit their scalability and economic feasibility in biogas production.

One approach, disclosed herein, to improving biogas production efficiency integrates specialized microbial consortia capable of degrading lignocellulosic materials under mesophilic, anaerobic conditions. Specific fungi within the genus Neocallimastigomycota are utilized for their enzymatic capacity to hydrolyze lignocellulose at moderate temperatures, approximately 40° C. This enzymatic action allows for the conversion of complex plant fibers into simpler compounds, which can then be digested by methanogenic microorganisms. Establishing a stable microbial culture optimized for plant material degradation within a bioreactor setting can thus reduce energy inputs required for pretreatment and enhance biogas yields, creating a resilient, high-yield system.

This approach further incorporates a secondary thermophilic hydrolysis stage, using organisms from the Caldicellulosiruptor genus and similar microorganisms, which supports the complete breakdown of residual biomass, including microbial biomass. This approach maximizes the production of digestible materials, minimizes reliance on chemical pretreatment, and optimizes methane output as a renewable energy source. The output from this second stage, which contains solubilized and partially digested organic compounds, can then proceed efficiently to a final stage of anaerobic digestion, converting the remaining material into biogas. This approach supports streamlined, high-efficiency renewable methane production and allows for separation of alternative byproducts, such as lignin-based materials, which may be repurposed for additional uses or valorization.

### SUMMARY

The present invention provides a multi-stage bioconversion system and method optimized for the efficient breakdown and conversion of lignocellulosic feedstocks into high-quality biogas, primarily methane, with minimized carbon dioxide content. The system includes sequential anaerobic bioreactors configured for targeted stages of anaerobic digestion, specifically designed to maximize substrate breakdown and ensure stability across various microbial communities.

The process begins with a low-moisture, mesophilic anaerobic secretome bioreactor (ASB1), which uses an inoculated microbiome derived from the microbiota of herbivore rumens. This inoculated microbial community, rich in lignocellulose-degrading organisms and associated methanogens, promotes biogas production, enzymatic hydrolysis, partial acidogenesis, and acetogenesis of lignocellulosic feedstock, breaking down complex polymers into simpler, soluble compounds and produces significant microbial biomass. ASB1 operates under mesophilic conditions to stabilize pH, and is designed to prevent inhibitory compound accumulation and reduce the need for extensive pretreatment steps. The effluent produced by ASB1, enriched with partially degraded organic compounds and substantial microbial biomass, is subsequently directed to a second thermophilic anaerobic secretome bioreactor (ASB2).

ASB2 further breaks down the ASB1 effluent through enzymatic hydrolysis, acetogenesis, and acidogenesis, while simultaneously pasteurizing the material to eliminate non-thermophilic microbes and pathogens. ASB2 operates under high-moisture, thermophilic conditions, maximizing the conversion of organic matter in the effluent of ASB1 into substrates suitable for methanogenesis. The effluent from ASB2 is optimized for the final-stage methanogenic digestion, producing a mix of methanogenic precursors with minimal inhibitory byproducts.

The methanogenic digestion stage (MD3) completes the conversion process by transforming volatile fatty acids and other intermediates into high-quality biogas, primarily methane. MD3 is optimized to minimize carbon dioxide production, producing an effluent rich in bicarbonate as a byproduct. This bicarbonate provides buffering capacity for earlier stages, enhancing process efficiency and stability across the system.

The invention supports a sustainable, high-yield approach to biogas production, with features that improve microbial stability, reduce pretreatment requirements, and enable modular scalability. By carefully controlling pH, moisture, temperature, and substrate exposure across each stage, the system efficiently converts lignocellulosic feedstock into a clean, energy-rich biogas with minimal environmental impact.

DESCRIPTION OF THE DRAWINGS

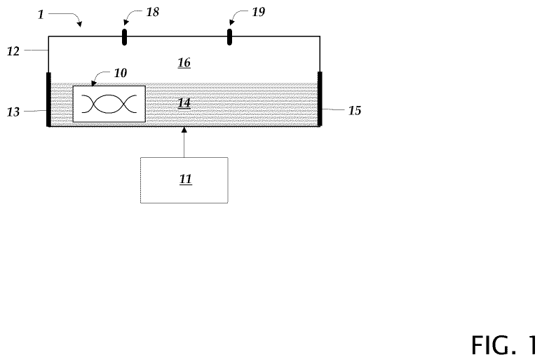

FIG. 1 illustrates an example of a first-stage anaerobic secretome bioreactor (ASB1).

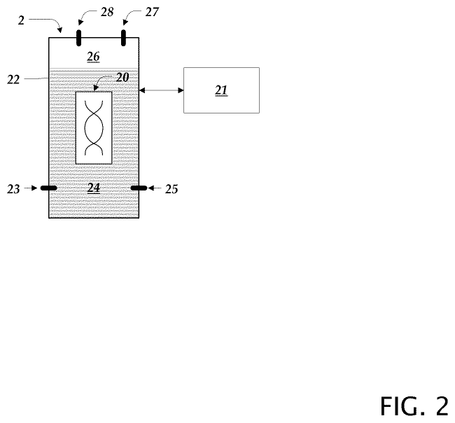

FIG. 2 illustrates an example of a second-stage anaerobic secretome bioreactor (ASB2).

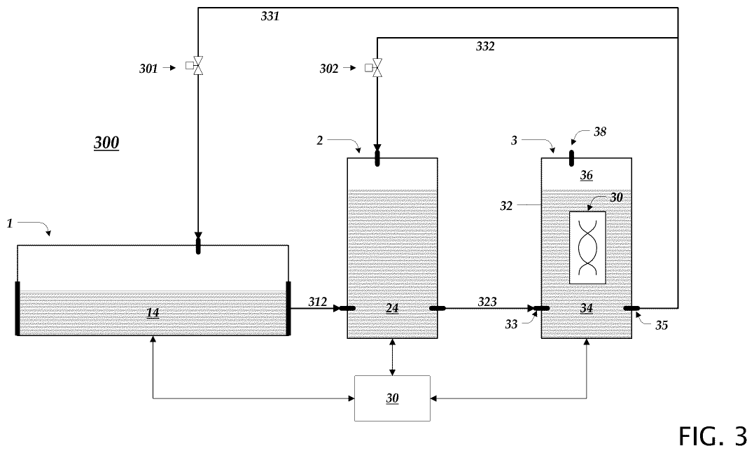

FIG. 3 illustrates an example of a three-stage system (300) that includes a third-stage methanogenic digester (MD3).

### DETAILED DESCRIPTION

### Terms Used in This Disclosure

The terms defined below, as used herein, may differ from conventional usage.

“Anaerobic secretome bioreactor” and “ASB” typically refers to bioreactors configured to facilitate anaerobic biological processes for the breakdown of feedstock substrates across various stages of the anaerobic digestion—hydrolysis, acidogenesis, acetogenesis, and, optionally, methanogenesis. These bioreactors operate under controlled anaerobic conditions and may be configured to process substrates with variable moisture content, accommodating both low-moisture (dry) and high-moisture (wet) environments. The term “secretome” refers to the bioreactor's reliance on microbial communities that produce and secrete enzymes (exozymes), which facilitate the degradation of complex organic matter and enhance substrate conversion efficiency throughout the distinct stages of anaerobic digestion.

“Biopolymers” typically refers to naturally occurring macromolecules produced by living organisms, including polysaccharides (e.g., starch, cellulose, and glycogen), proteins (composed of amino acids), nucleic acids (such as DNA and RNA), and structural materials like chitin and lignocellulose. This term also includes microbial biopolymers, such as microbial cell walls, polyhydroxyalkanoates (PHAs), alginate, and xanthan gum, which are synthesized by various microorganisms.

“Community” in the context of microorganisms and digestion typically refers to a cooperative assembly of diverse microbial species that work together synergistically to decompose complex organic matter into simpler compounds.

“Endozymes” (also known as endoenzymes or intracellular enzymes) typically refers to the enzymes that function within the cells of microorganisms, such as bacteria and fungi. These enzymes catalyze internal metabolic reactions, converting substrates into cellular Gibbs energy (e.g., ATP (Adenosine Triphosphate)), synthesizing cellular components, and producing metabolic byproducts—each essential for the organism's growth, maintenance, and overall function. This contrasts with exozymes, which are either secreted into the surrounding environment or bound to the external cell membrane, where they operate outside the microbial cell to break down complex organic materials.

“Exozymes” (also known as exoenzymes or extracellular enzymes) typically refers to enzymes that are either secreted by microorganisms, such as bacteria and fungi, into their surrounding environment or bound to the outer cell membrane, where they catalyze the breakdown of complex organic materials outside the microbial cell. This contrasts with endozymes, which function inside the cell to catalyze metabolic reactions within the organism itself.

“Extracellular enzyme system” typically refers to the collection of enzymes that are synthesized by microorganisms, such as bacteria and fungi, and either secreted outside of the cell or anchored to the outer cell membrane. These enzymes function externally to catalyze the breakdown of complex organic compounds into simpler, more accessible substrates, producing intermediate products, such as sugars, fatty acids, and amino acids. In the context of anaerobic digestion, these breakdown products are essential substrates for other microbial communities, facilitating further conversion into biogas and enhancing overall system efficiency.

“Feedstock” typically refers to the raw organic materials introduced into a digester to be broken down by microorganisms during the digestion process. Feedstocks provide the source of carbon and nutrients necessary for microbial activity, ultimately leading to the production of biogas and digestate.

“FOG” typically refers to Fats, Oils, and Grease, which are organic compounds commonly present in wastewater, food waste, and industrial byproducts. FOG is primarily composed of glycerides, free fatty acids (FFAs), and glycerol. In anaerobic systems, FOG can create operational challenges, such as scum formation, clogging of equipment, and inhibition of microbial processes, particularly due to the accumulation of long-chain fatty acids (LCFAs). Despite these challenges, FOG is a highly energy-dense material and, when properly managed, can significantly enhance biogas production.

“Inoculated microbiome” typically refers to a microbial community deliberately introduced into or established within a bioreactor to initiate and sustain biological processes, including the breakdown of feedstocks and other substrates. In one example, this microbiome originates from a microbiota.

“Intracellular enzyme system” refers to the collection of enzymes that are synthesized and remain within the cell of microorganisms, such as bacteria and fungi. These enzymes function internally to catalyze metabolic processes that convert substrates into cellular Gibbs energy (e.g., ATP (Adenosine Triphosphate)) and cellular components necessary for growth and maintenance. As a result of these metabolic processes, waste products such as carbon dioxide, ammonia, organic acids, and hydrogen gas are produced. In the context of anaerobic digestion, these waste products are valuable intermediates, as they serve as substrates for other microbial communities, enhancing biogas production and overall digestion efficiency.

“Lignocellulose” typically refers to plant biomass as recognized within the industry, composed of lignin, cellulose, hemicellulose, and other biopolymers. These materials include various forms of green waste and energy crops, which comprise the primary structural components of plant cell walls, along with cell contents (e.g., lipids, proteins, nucleic acids, and other cytoplasmic molecules and organelles or their components). Examples of lignocellulosic material sources include, but are not limited to, wood, grasses, agricultural residues (such as corn stover and straw), green waste, forest residues, energy crops (e.g., switchgrass, miscanthus), municipal solid waste, and certain industrial by-products.

“Mesophilic” usually refers to processes or microorganisms that operate optimally at moderate temperatures, typically between 30° C. to 40° C. (86° F. to 104° F.). However, in the context of this invention, “mesophilic” typically refers to temperatures ranging from about 20° C. and 55° C. (68° F. to 131° F.), essentially spanning the transition zone between conventional psychotropic and thermophilic conditions.

“Methanogenic digestion” typically refers to a stage of biogas

production—methanogenesis—where methanogenic archaea convert soluble organic compounds into methane, along with carbon dioxide and trace gases depending on the compounds.

“Microbe” typically refers to a microorganism, which is a microscopic living organism. The term encompasses a variety of life forms, including bacteria, archaea, fungi, and protozoa. While “microbe” is often used interchangeably with “microorganism,” it is a more informal term commonly used in discussions about ecology, health, and biotechnology.

“Microbiota” typically refers to the diverse community of microorganisms in a specific environment, such as the digestive systems of large herbivores. Such communities, typically comprising bacteria, archaea, fungi, and protozoa, contribute to the breakdown and digestion of plant material and other organic matter.

“Scum” typically refers to a layer of solid or semi-solid material that floats on the surface of a digester due to the accumulation of substances like fats, oils, grease (FOG), fibrous materials, or gas bubbles trapped in organic matter.

“Sludge” typically refers to the semi-solid byproduct that results from the treatment of wastewater, industrial effluents, or other organic materials. Sludge is primarily composed of water, organic matter, microorganisms, and various inorganic particles.

“Soluble products” and the like typically refer to various compounds generated during the breakdown of lignocellulosic materials and/or other feedstocks that are capable of dissolving in a liquid phase. These products generally include smaller molecular components derived from the hydrolysis or decomposition of structural biomass (e.g., cellulose, hemicellulose, lignin) and non-structural organic constituents (e.g., cell contents and other organic matter) present in the feedstocks. Examples of such feedstocks include municipal waste, agricultural residues, and industrial by-products. In certain contexts, including this invention, soluble products may also encompass suspended microbial cells, macromolecules, and micelles in the liquid phase.

“Substrate” typically refers to organic material that serves as a reactant in metabolic processes carried out by individual microorganisms or within a microbial community at any stage of a digestion process, anaerobic or otherwise. Substrates may include feedstocks, partially digested materials, and effluents from other processing stages. These substrates provide the nutrients and cellular Gibbs energy (e.g., ATP (Adenosine Triphosphate)) required for microorganisms to break down complex organic matter into simpler compounds, supporting both extracellular reactions in a digester environment and enzymatic reactions within cells.

“Symbiotic” in the context of microbial species typically refers to bacteria, fungi, and/or other microorganisms that coexist within microbial communities, such as those in the digestive systems of large herbivores or in bioreactors, where they assist in the digestion of lignocellulosic materials, biopolymers, and other organic substrates. These microorganisms often establish mutualistic relationships within their environment—enhancing the breakdown and metabolism of complex organic matter—while benefiting from the stability and nutrient-rich conditions provided by the host organism or the controlled bioreactor setting.

“Synthetic” in the context of microbial communities typically refers to a deliberately designed and engineered community of microorganisms that collaborate to optimize the breakdown of complex organic materials, particularly under anaerobic conditions. Unlike natural microbial communities, which evolve without human intervention, synthetic microbial communities are custom-tailored to achieve targeted outcomes, such as enhancing the efficiency of organic waste degradation, maximizing biogas production, and/or mitigating process inhibitors.

“Thermophilic” usually refers to processes or microorganisms that operate optimally at high temperatures, typically between 50° C. and 70° C. (122° F. to 158° F.). However, in the context of this invention, “thermophilic” refers to higher temperatures, specifically ranging from about 65° C. to 85° C. (149° F. to 185° F.), essentially spanning the transition zone between conventional thermophilic and hyperthermophilic conditions.

“WAS” typically refers to Waste Activated Sludge, which is the biomass consisting of microbial cells and organic matter removed from the aeration stage of wastewater treatment plants. WAS is a byproduct of the activated sludge process, where microorganisms metabolize organic pollutants in wastewater. Once the biological treatment is complete, the excess microbial biomass, or WAS, is separated from the treated water. In certain contexts, including this invention, the term WAS may also encompass primary sludge (e.g., from sedimentation tanks), mixtures of primary sludge and WAS, anaerobic digestion solids, and other mixtures from wastewater treatment plants.

### Processing Stages Versus Digestion Stages

Two distinct types of “stages” are discussed in this disclosure—processing stages and digestion stages. “Processing stages” as used herein refer to the sequential arrangement of distinct physical units or bioreactors within a multi-stage biogas production system. Each unit is dedicated to specific anaerobic digestion functions or environmental conditions that facilitate substrate breakdown. For example, “ASB1→ASB2→MD” indicates a system where a first Anaerobic Secretome Bioreactor performs an initial processing stage—including some biogas production, transferring degraded substrate to a second Anaerobic Secretome Bioreactor for further processing, and finally to a Methanogenic Digester for biogas production. Additionally, each processing stage may have unique operational characteristics, such as moisture levels, operating temperature, pH, microbial community composition, and other parameters.

In contrast, “digestion stages” as used herein refer to the biochemical phases within anaerobic digestion that sequentially transform organic material into simpler compounds and ultimately biogas. These stages include hydrolysis, acidogenesis, acetogenesis, and methanogenesis. Each digestion stage represents a distinct biochemical transformation and may occur within one or across multiple physical processing stages, depending on system design. For instance, in the example above, both ASB1 and MD may contribute to produce biogas.

### PROCESSING STAGE 1

### ASB1 Lignocellulose Degradation Technologies

In one example, the disclosed biogas production technologies employ an initial processing stage (ASB1) involving microbial hydrolysis, specifically designed to maximize the conversion of lignocellulosic feedstocks into soluble compounds, some biogas, and microbial biomass. This process typically begins with a mesophilic hydrolysis phase, in which lignocellulosic materials—such as green waste, agricultural residues, or energy crops—are degraded by a microbial community that includes fungi from the genus Neocallimastigomycota. These fungi break down lignocellulose through two complementary mechanisms: (1) physically disrupting the lignin matrix by extending their mycelia into plant structures, and (2) secreting extracellular enzymes (a secretome) that hydrolyze cellulose, hemicellulose, and lignin into soluble sugars and other compounds under moderate, anaerobic conditions, typically around 40° C. This initial stage reduces the need for costly and energy-intensive thermal or chemical pretreatment by breaking down complex plant fibers into simpler, metabolizable components.

ASB1 is configured to operate with a water content of approximately 1 to 15 percent by volume. By maintaining the bioreactor environment at a temperature within the mesophilic range and a pH in the range of 6.5 to 8.0, the fungi and associated microbial community can effectively establish and sustain a stable culture. Enzymes produced by Neocallimastigomycota fungi hydrolyze lignin, exposing cellulose and hemicellulose, which are further broken down by exozymes into simpler molecules, such as sugars and organic acid anions, some of which may be converted into biogas by associated methanogens. ASB1 facilitates initial acidogenesis, generating organic acids primarily in their anionic forms (e.g., acetate, propionate), which serve as precursors for methanogenesis. While methanogenesis by methanogenic microorganisms is limited at this stage, it supports the growth and activity of Neocallimastgomycota. Full conversion of substrates in the first-stage effluent into methane typically occurs in downstream processing stages specifically configured to optimize methanogenesis. In some examples, undigestible fibrous materials, such as lignin, are removed from the first stage effluent before it is transported to the second stage.

After the mesophilic hydrolysis stage, the partially degraded substrate typically undergoes a secondary hydrolysis stage in a thermophilic environment. This processing stage (ASB2) employs thermophilic organisms to further hydrolyze resistant biomass, including microbial biomass and any remaining lignocellulose, cellulose, and hemicellulose. Operating at a temperature range of 65° C. to 85° C. and a pH of 6.5 to 8.0, these thermophilic conditions enhance the breakdown of lignocellulosic materials as well as active and dormant microbial cells present in the substrate from ASB1. This second stage produces a liquid effluent rich in solubilized organic compounds, largely free of lignin and pathogens, making it highly suitable for final methanogenic digestion.

The liquid effluent from the thermophilic hydrolysis stage is directed into a final methanogenic digestion stage (MD), where methanogenic microorganisms convert the remaining digestible materials into biogas, maximizing methane production and overall energy yield. By integrating mesophilic and thermophilic hydrolysis stages, this multi-stage approach minimizes the need for chemical additives, enhances scalability, and offers an economically viable solution for renewable methane generation from lignocellulosic biomass.

### ASB1 Inoculum

In some examples, the microbiota utilized in ASB1 are selected for their robust ability to degrade lignocellulosic materials under anaerobic conditions. This microbial community is primarily sourced from the digestive systems of large herbivores, where a diverse community of lignocellulose-degrading microorganisms has evolved to efficiently process complex plant fibers. Notably, fungi from the genus Neocallimastigomycota are key contributors within this community. These fungi thrive in anaerobic environments and produce a secretome rich in exozymes, which can effectively hydrolyze lignocellulose, cellulose, hemicellulose, and other complex polysaccharides into simpler, soluble sugars at moderate temperatures, typically around 40° C.

The microbiota includes not only lignocellulose-degrading fungi but also symbiotic bacteria and archaea, which play supportive roles in establishing a stable anaerobic environment conducive to substrate breakdown. These microbes are well-suited for the bioreactor environment, where they function cooperatively to maximize cellulose and hemicellulose degradation, enhancing the substrate's availability for further processing in subsequent stages. By introducing this naturally adapted microbiota into the mesophilic bioreactor ASB1, the system effectively mimics the digestive processes of large herbivores, optimizing the conversion of lignocellulosic material into compounds and other materials suitable for biogas production.

The microbiota used in ASB1 is derived from the forestomach of one or more large herbivores such as cows, sheep, and goats. The rumen, as the largest compartment of the forestomach, functions as a specialized fermentation chamber, maintaining an anaerobic environment that is ideally suited for breaking down fibrous plant material. This part of the digestive system hosts a highly diverse microbial community—including bacteria, archaea, fungi, and protozoa—adapted to decompose lignocellulosic materials through enzymatic hydrolysis.

By sourcing the microbiota from the rumen (and optionally other chambers of the forestomach), ASB1 is inoculated with a naturally adapted microbial consortium capable of producing abundant cellulases, hemicellulases, and other exozymes necessary for breaking down the structural components of plant biomass. After inoculation, the rumen-derived microbiome establishes itself within ASB1, replicating the conditions needed to initiate and sustain efficient lignocellulose degradation.

This synergistic setup enhances the reactor's ability to maintain efficient lignocellulose degradation, as the inoculated microbiome helps sustain fungal viability and activity over time. By creating a balanced anaerobic environment, the inoculated microbiome mitigates potential disruptions in pH, redox potential, and nutrient availability that could otherwise hinder fungal survival. Additionally, certain bacteria and protozoa produce growth factors, vitamins, and metabolites that support fungal health, enabling the fungi to continue producing essential cellulases, hemicellulases, and other exozymes for extended periods. The inoculated microbiome's natural adaptation to lignocellulosic digestion further reinforces the fungi's stability, making the system resilient to fluctuations and ensuring prolonged enzymatic activity in ASB1.

### ASB1 Inoculation

In some examples, effective digester inoculation is essential for establishing a stable and efficient community capable of degrading feedstocks and other substrates. By introducing a carefully selected microbiota, in some examples, the digester gains a robust community of bacteria, archaea, protozoa, and fungi. This inoculated microbiome, characterized by species interactions that facilitate substrate breakdown, supports continuous anaerobic processing and can be introduced in various ways to optimize performance based on feedstock stability and system requirements.

In general, the inoculated microbiome introduced into ASB1 establishes a stable and synergistic community. The community produces a range of exozymes, some secreted into the environment and others remaining cell-bound, which catalyze the degradation of lignocellulosic material under mesophilic conditions at approximately 40° C. This moderate temperature optimizes both energy efficiency and microbial activity. Through this enzymatic process, complex polymers in the plant material are broken down into simpler compounds, while microbial biomass and soluble intermediates are diverted into the effluent for processing in later stages. Biogas is also produced.

In one example, microbiota and any supplements typically only need only be introduced once, as the initial inoculation establishes a robust community capable of sustaining itself under stable operating conditions. This one-time inoculation forms a self-maintaining ecosystem within ASB1, where microbial species achieve a balanced interaction that drives continuous substrate degradation and biogas production. Once stabilized, the community's diversity and syntrophic interactions enable it to adapt to minor feedstock variations, supporting steady-state operation without the need for further inoculation. This approach is particularly advantageous in systems with relatively uniform feedstock inputs, allowing for consistent microbial activity and energy efficiency over extended periods.

In another example, periodic inoculation of the feedstock may be employed to maintain or enhance the inoculated microbiome within ASB1. In this approach, microbiota and any supplements are introduced at regular intervals to replenish or boost the community, ensuring that enzymatic activity remains high and that microbial populations capable of degrading specific feedstock components and biogas production are maintained. Periodic inoculation can be particularly useful when feedstock composition varies over time, allowing the community to adapt to changing substrate profiles and continue efficient degradation.

Alternatively, continuous inoculation may be employed, where inoculated microbiome is gradually introduced, such as with feedstock. This method creates a dynamic microbial environment, ensuring a steady influx of active microbes that can rapidly respond to fluctuations in substrate concentration and composition. Continuous inoculation supports sustained enzyme production and maintains optimal microbial interactions, enhancing the resilience of the community. This approach can be particularly advantageous in high-throughput systems where consistent biogas production and substrate conversion are essential. Both periodic and continuous inoculation methods offer flexibility in managing the microbial ecosystem, allowing operators to tailor inoculation strategies based on feedstock variability and specific operational goals.

### Challenges with Mixed Feedstocks

In agricultural and mixed organic waste streams, the presence of fats, oils, and grease (FOG) and waste activated sludge (WAS) presents unique challenges for anaerobic digestion processes. FOG, commonly derived from food processing and municipal waste, and WAS, a byproduct of wastewater treatment, are energy-rich components. However, they can disrupt digestion when introduced into conventional agricultural feedstocks, such as crop residues or manure. FOG's high lipid content can create physical barriers within digesters, leading to scum formation and reduced mass transfer, while WAS introduces nitrogen-rich proteins that may contribute to ammonia buildup, impairing microbial function. These factors can significantly impact digestion efficiency, stability, and biogas yield, making it critical to effectively manage these components within mixed feedstocks.

Including FOG and WAS in feedstocks can increase biogas potential due to their energy density but requires careful management to avoid inhibitory effects on microbial activity. FOG can lead to the accumulation of long-chain fatty acids (LCFAs), which are challenging to degrade and can inhibit methane-producing microbes. WAS, while contributing essential microbial biomass and nutrients, can increase ammonia levels due to its protein content, potentially leading to ammonia toxicity within a biodigester. The combination of FOG and WAS with conventional feedstocks requires careful management to balance nutrient loads and prevent inhibitory conditions, ensuring that digestion remains efficient and stable.

Co-digestion of complementary feedstocks can be particularly advantageous due to the co-occurrence of materials. For example, green waste can be digested with WAS and FOG, bottoms from corn ethanol facilities with corn stover, and cattle manure with energy crops. In some examples, WAS, FOG, fermentation bottoms, or manure may either be mixed directly with the primary feedstock into ASB1 or combined with ASB1 effluent for further processing in ASB2.

### Optimizing with Supplemental Microbial Community

To address these challenges, the inoculated microbiome in ASB1 may be supplemented with specific microorganisms that enhance degradation efficiencies and mitigate operational issues. For example, lipase-producing microbes facilitate the breakdown of FOG by hydrolyzing lipids into glycerol and free fatty acid anions (FFAAs). This process mitigates scum formation and prevents the accumulation of LCFAs, which can inhibit microbial activity and disrupt system performance.

In various examples, supplementation may also include cellulolytic microbes to enhance lignocellulose breakdown or hydrogenotrophic methanogens to improve biogas quality by converting hydrogen into methane. Such microorganisms may be introduced in ASB1 and/or ASB2, depending on the specific process requirements and feedstock composition.

The following thermophilic organisms have been identified as suitable for FOG degradation in thermophilic anaerobic environments, such as ASB2, where high temperatures accelerate lipid hydrolysis and microbial activity. Microorganisms with similar functions may additionally or alternatively be identified for mesophilic anaerobic environments, such as ASB1, to optimize performance under moderate-temperature conditions.
1. Bacillus thermocatenulatus: A thermophilic bacterium known for its robust lipase production, particularly effective at temperatures between about 65° C. and 85° C. This microorganism hydrolyzes a wide variety of glycerides—including triglycerides, diglycerides, and monoglycerides—and is suitable for breaking down complex lipid compounds in the ASB environment.
2. Geobacillus stearothermophilus: This bacterium thrives at temperatures between about 65° C. and 85° C. and is capable of producing thermostable lipases that degrade fats and oils. The enzyme activity of this organism makes it highly effective in breaking down LCFAs, a key component of FOG.
3. Thermomyces lanuginosus: A thermophilic fungus that produces thermostable lipases. It is widely used in industrial applications for FOG degradation due to its ability to operate at high temperatures, making it ideal for thermophilic environments. The lipases secreted by this fungus can efficiently hydrolyze glycerides—including triglycerides, diglycerides, and monoglycerides—into glycerol and fatty acids.
4. Anaerobacillus thermoterrificus: This thermophilic bacterium produces extracellular lipases that effectively break down lipids at temperatures of about 65° C. and 85° C., converting them into fatty acids and glycerol, which can then be efficiently metabolized by anaerobic microorganisms in subsequent digestion stages.
5. Clostridium spp.: A thermophilic bacterium that converts glycerol and other hydrolysis products into VFAs (such as acetic acid and butyric acid), which are crucial intermediates for biogas production.

In parallel, nitrogen-assimilating microbes can be introduced to manage the ammonia produced from WAS, stabilizing nitrogen levels and preventing ammonia toxicity. This targeted supplementation not only helps to maintain optimal microbial function and balance within the mesophilic environment but also enhances biogas yields by optimizing the breakdown of complex substrates from diverse feedstocks.

The following thermophilic microorganisms have been identified as suitable for efficient WAS degradation in thermophilic anaerobic environments, such as ASB2, where high temperatures accelerate lipid hydrolysis and microbial activity. Microorganisms with similar functions may additionally or alternatively be identified for mesophilic anaerobic environments, such as ASB1, to optimize performance under moderate-temperature conditions.
1. Thermoanaerobacterium thermosaccharolyticum: Known for its ability to produce exozymes that break down complex carbohydrates and proteins, this bacterium thrives at thermophilic anaerobic conditions and contributes to the degradation of organic material present in WAS.
2. Anaerobacillus thermoterrificus: This thermophilic bacterium produces exozymes, including proteases and lipases, which are essential for breaking down proteins and lipids in WAS in thermophilic anaerobic conditions.
3. Geobacillus stearothermophilus: A highly effective thermophile capable of producing thermostable proteases and lipases. This microorganism is particularly useful for degrading the lipid and protein content in WAS, contributing to the breakdown of cellular debris from activated sludge.
4. Thermoanaerobacter spp.: These bacteria are known for their robust metabolic activity in thermophilic anaerobic conditions, contributing to the degradation of polysaccharides, proteins, and lipids present in WAS.
5. Clostridium spp.: A thermophilic bacterium that converts glycerol and other hydrolysis products into VFAs (such as acetic acid and butyric acid), which are crucial intermediates for biogas production.
6. Caldicellulosiruptor spp.: These thermophilic anaerobes are particularly well suited to hydrolysis and solubilization of lignocellulose and microbial cell walls, including those found in corn fermentation bottoms and cattle manure.

### Example Stage 1 Anaerobic Secretome Bioreactor

In one example, an initial stage anaerobic secretome reactor, such as ASB1 as illustrated in FIG. 1, includes a digester vessel, such as vessel 12, and is configured to break down feedstock or other substrate, such as feedstock 14. The vessel includes space, such as space 16, to allow for the accumulation and removal of gases produced during substrate digestion. Vessel 12 may be equipped with a thermally insulated chamber to maintain the temperatures required for efficient mesophilic degradation. A temperature control system ensures uniform temperatures throughout the vessel, supporting consistent enzyme activity and stable microbial function. This setup is optimized for processing low-moisture substrates to facilitate the thorough breakdown of complex organic compounds under mesophilic, anaerobic conditions.

Vessel 12 may include at least one port, such as port 18, configured to allow gas or other substances to vent, exit, or be collected from within. Additionally, at least one port, such as port 19, may allow water, other fluids, buffering agents, or additives to be introduced into the vessel's interior. The vessel may also include at least one feed port, such as port 13, for introducing feedstocks or other substrates, and at least one discharge port, such as port 15, for discharging liquid and/or solid effluents. Additional ports suitable for similar or alternative purposes may be included for similar or alternative functions as needed, with positioning and configuration tailored to optimize ASB1 performance and accessibility.

ASB1 may include an internal stirring or mixing mechanism(s), such as mechanism 10, to ensure even distribution of the feedstock, enhancing contact between the microbial community and substrate within the reactor. For example, a mechanical agitator or paddle system may be used to gently shift the substrate, promoting consistent exposure of solid material to microbial enzymes and ensuring an even distribution of moisture. Additional or alternative mixing mechanisms may also be employed as needed. These mixing capabilities facilitate uniform hydrolysis, promoting efficient degradation of lignocellulosic materials and other feedstocks within the low moisture reactor environment.

ASB1 may further benefit from a pH monitoring and control system(s), such as system 11, to maintain an optimal pH range conducive to mesophilic microbial activity and enzyme function. These systems can include pH sensors strategically positioned within the vessel to provide continuous, real-time measurements of the substrate environment. When pH fluctuations are detected, buffering agents—such as bicarbonate or other suitable alkaline or acidic additives—can be automatically introduced through a controlled dosing mechanism. In some cases, effluent from another stage, such as a methanogenic digestion stage of processing, may be repurposed as a buffering agent, enhancing both process efficiency and sustainability. This pH control capability helps maintain a stable reactor environment, typically within a pH range of 6.5 to 8.0, preventing acidification that could inhibit microbial activity and enzyme function.

Vessel 12 may be oriented in any suitable position, such as vertical, horizontal, or angled, depending on specific design requirements and operational goals of the anaerobic digestion process. Orientation can influence factors such as feedstock flow dynamics, mixing efficiency, gas-liquid separation, as well as feeding and discharge convenience. For example, a horizontal orientation may optimize dry substrate flow, while a vertical orientation could promote stratification of gas, liquid, and solid phases. The selected orientation should facilitate optimal retention times, degradation efficiency, and convenient feeding, discharge, operation, maintenance, and monitoring.

### ASB1 Characteristics

In one example, ASB1 is configured to create and maintain specific internal conditions essential for efficient mesophilic anaerobic digestion. Three key controlled parameters—moisture level, pH, and temperature—are managed to support the optimal activity of the inoculated microbiome responsible for breaking down lignocellulosic and other substrates. Each of these parameters is critical to maintaining a stable and balanced microbial environment within ASB1. Moisture levels are carefully regulated to support microbial activity while conserving energy, with low-moisture content tailored to the needs of a dry anaerobic system. pH is maintained within a range that minimizes microbial inhibition, stabilizes enzyme function, and fosters optimal conditions for substrate degradation and methane production. Additionally, a mesophilic temperature range is controlled to balance microbial activity with energy efficiency, promoting steady substrate degradation and biogas yields.

### ASB1 Moisture Levels and Sources

In one example, ASB1, a horizontal dry reactor, operates with moisture levels ranging from approximately 1% to 15%. This range creates an ideal environment for high-fiber materials such as green waste, agricultural residues, or energy crops. By maintaining low moisture content, the ASB optimizes energy utilization efficiency and fosters conditions that support the activity and stability of the microbial community, particularly lignocellulose-degrading fungi from the genus Neocallimastigomycota and their symbiotic counterparts.

Minimizing the flow of liquid through ASB1 offers additional advantages. It maximizes the concentration of digestible materials, enhancing microbial access and digestion efficiency. Furthermore, it reduces energy costs associated with heating the material to thermophilic temperatures. Together, these factors contribute to improved process sustainability and cost-effectiveness.

Water and/or other fluids may be introduced into the vessel to achieve optimal moisture levels. In one example, fluid is added at or near the discharge end to flow counter to the movement of the feedstock, gently flushing portions of the microbial community, including fungal zoospores, toward the feedstock inlet. This reverse flow helps inoculate the incoming feedstock with active microbial agents, including zoospores, which can readily colonize newly introduced substrate material. By encouraging microbial distribution, the flow promotes colonization and microbial activity throughout the substrate path while also maintaining ideal moisture conditions for effective digestion.

Alternatively, or additionally, water or other fluids may be applied through a series of spray nozzles positioned along a portion of the vessel's length to distribute moisture directly onto the substrate. This spray system can be adjusted to target specific areas as needed, ensuring that the substrate maintains sufficient hydration without excessive pooling. By creating a controlled moisture gradient, the fluid spray promotes microbial activity across the substrate, supporting consistent degradation and efficient biogas production throughout the vessel.

Alternatively, or additionally, effluent from a prior or subsequent processing stage may be introduced into the vessel to enhance both moisture content and microbial activity. This effluent, particularly if rich in bicarbonate, helps maintain optimal pH levels and provides essential buffering capacity to support stable microbial growth and enzymatic activity. Recycling liquid effluent, which may carry active microorganisms and beneficial nutrients, improves overall process efficiency, fostering a well-balanced microbial community that enhances substrate breakdown and biogas production.

Alternatively, or additionally, a substrate or substrate supplement with high water content, such as waste-activated sludge (WAS), may be introduced to the vessel to enhance moisture levels naturally. Incorporating high-moisture materials like WAS not only increases the overall hydration of the substrate, aiding microbial activity, but also provides additional nutrients that support a balanced microbial community. This approach maintains the desired moisture content without direct water addition, fostering optimal conditions for microbial colonization and efficient substrate breakdown.

### ASB1 pH Management and Stability

In one example, to optimize soluble intermediate production, ASB1 is maintained within an optimal pH range of approximately 6.5 to 8.0. This range supports the metabolic activities of the inoculated microbiome, including bacteria and fungi that contribute to breaking down complex organic substrates into simpler compounds. Maintaining this pH range promotes a balanced microbial community, enabling each microbial group to efficiently perform its role in substrate degradation, thereby enhancing production of soluble compounds.

To control pH, buffering agents like bicarbonate may be introduced, either through liquid effluents or direct supplementation, to counteract potential pH drops. Regular pH monitoring allows for timely adjustments, ensuring the microbial community remains balanced and resilient. Maintaining pH within this optimal range creates an environment conducive to efficient substrate degradation in ASB1.

### ASB1 Temperature Management

In one example, ASB1 operates within a controlled mesophilic temperature range, typically maintained between approximately 30° C. and 40° C. This temperature range optimally supports the metabolic activities of the microbial community involved in the initial stages of processing. Mesophilic conditions achieve a balance between microbial activity and energy efficiency, allowing the inoculated microbiome to thrive and work synergistically. Maintaining this temperature range enhances enzyme production and promotes the breakdown of complex substrates, such as cellulose and lignin, into simpler compounds. Most of these compounds are further converted by methanogens into biogas in ASB1 and/or in a downstream processing stage.

Mesophilic temperatures also enhance system resilience by supporting a diverse microbial community capable of degrading lignocellulosic and other substrates under low-moisture conditions. This stability enables ASB1 to achieve consistent degradation yields while minimizing the heating costs associated with higher temperatures. Additionally, mesophilic conditions reduce the risk of thermal stress on microbial populations, fostering a robust microbial community that can adapt to variations in feedstock composition. By maintaining temperatures within this mesophilic range, ASB1 promotes optimal microbial performance, ensuring efficient substrate degradation and steady feedstock degradation throughout the bioreactor.

### ASB1 Substrate Breakdown

In one example, ASB1 is configured to maximize the exposure of lignocellulosic feedstock to the inoculated microbial community, promoting efficient enzymatic hydrolysis under low-moisture, mesophilic conditions, typically maintained at a temperature range of approximately 30° C. to 40° C. and a pH between 6.5 and 8.0. The stable and synergistic microbial community adapts effectively to these conditions, producing a variety of exozymes—both secreted and cell-bound, such as cellulases, hemicellulases, and ligninases—that collaboratively break down the structural components of the feedstock, transforming complex polymers into simpler, soluble compounds. Following enzymatic hydrolysis, a portion of these simpler compounds may be metabolized by the microbial community within ASB1, producing biogas, while the remaining soluble compounds and suspendable materials are transported in the effluent for downstream processing.

During processing, the close mixing of solid and liquid phases within ASB1 creates a cohesive semi-solid matrix, with hydrolyzed compounds and microbial biomass distributed throughout the feedstock. This intermingled matrix ensures effective contact between enzymes and substrates, supporting continuous breakdown of complex structural components, such as cellulose, hemicellulose, and lignin.

As the effluent exits ASB1, it primarily comprises a concentrated mix of partially degraded organic compounds, including volatile fatty acid anions, residual simple sugars, and other soluble byproducts, along with microbial biomass in both active and sporulated forms, as well as residual lignin and unconverted fiber materials. After removing undigestible fibrous materials, primarily lignin, ASB1's semi-solid effluent is well-suited as a substrate for further anaerobic digestion in subsequent stages. Removing lignin minimizes the risk of inhibition in downstream stages, while maintaining this integrated structure ensures that all digestible material remains accessible for downstream microbial activity. This staged approach maximizes bioconversion yield and efficiency.

### ASB1 Microbial Biomass Cultivation

The microbial biomass in ASB1 is not merely incidental but is deliberately cultivated as an integral component of the process. This biomass includes active microbial consortia and sporulated forms, which enhance the efficiency and robustness of downstream stages. Among these, only microorganisms tolerant of thermophilic conditions contribute directly to the ongoing degradation of organic substrates in ASB2. Active microbes drive hydrolysis and generate methanogenic precursors, while sporulated forms ensure a resilient microbial population capable of adapting to changes in feedstock composition or operating conditions. Additionally, microbial biomass serves as a readily degradable substrate in subsequent anaerobic digestion stages. By releasing intracellular enzymes, proteins, and other compounds, it drives methanogenesis and accelerates the breakdown of complex substrates. This deliberate cultivation strategy improves system performance by increasing methane yields and maintaining a stable, self-sustaining microbial ecosystem.

The microbial biomass from ASB1 that cannot tolerate the thermophilic conditions of ASB2 undergoes natural degradation during the second phase. Elevated temperatures in ASB2 induce thermal stress, causing non-thermophilic microorganisms to undergo cell lysis. This breakdown releases intracellular components, including enzymes, proteins, lipids, and nucleic acids, which become readily degradable substrates for thermophilic microbial communities. These released compounds enhance the production of methanogenic precursors, supporting efficient methane generation in downstream stages such as MD3.

In addition to cell lysis, some components of the biomass, such as resistant cell walls and organic matter, may remain inert during ASB2. These materials are either carried over to subsequent stages for further anaerobic degradation or persist as residual solids that may be separated from the process. Even so, inert biomass can act as a slow-release carbon source, gradually providing nutrients to sustain microbial activity. Furthermore, the thermophilic environment of ASB2 conditions certain components of the non-thermophilic biomass, breaking down weaker structures and increasing nutrient availability for active thermophilic microorganisms.

This staged degradation approach ensures that most microbial biomass from ASB1 contributes value to the system, either through direct activity or as a source of substrates and nutrients. By leveraging processes such as cell lysis, slow-release carbon contributions, and thermophilic conditioning, the system maximizes bioconversion yield and methane production efficiency. This integration of microbial biomass cultivation and utilization not only improves biogas yield but also supports a robust and adaptable anaerobic digestion process. These advancements enhance the system's sustainability and operational flexibility, ensuring consistent performance across diverse feedstock compositions.

### ASB1 Carbon and Nitrogen Management

In one example, maintaining a balanced carbon-to-nitrogen (C/N) ratio is critical in ASB1 to control the production of inhibitory compounds, such as ammonia and organic acids, and to support stable microbial activity. A C/N ratio typically between 20:1 and 30:1 provides sufficient carbon for microbial energy needs and nitrogen for growth, minimizing the risk of excessive ammonia accumulation from nitrogen-rich feedstocks. This balanced ratio helps limit the conversion of nitrogen compounds into ammonia while avoiding excess carbon that could lead to organic acid buildup.

Additionally, maintaining a pH between 6.5 and 8.0 further supports microbial stability by ensuring that organic acids remain primarily in their less inhibitory, anionic forms. This pH range reduces the toxicity risk from organic acids, promoting syntrophic relationships among microbial species.

Finally, selecting and blending feedstocks to achieve a balanced supply of carbon and nitrogen helps maintain the C/N ratio in ASB1. By controlling this balance, the reactor optimizes ammonia and organic acid levels, enhancing microbial resilience. As a result, ASB1 sustains a healthy microbial community with minimal need for external buffers or additional pretreatment methods, supporting efficient and cost-effective feedstock degradation.

### ASB1 Ammonia Management

In one example, ammonia can act as a digestion inhibitor in ASB1. Produced during the breakdown of nitrogen-rich compounds, such as proteins in the feedstock, ammonia accumulates in the reactor environment. Such ammonia typically exists in two forms: ammonium ions (NH₄⁺) and free ammonia (NH₃), with free ammonia being more toxic due to its ability to diffuse across microbial cell membranes, disrupting cellular functions. The balance between these forms is influenced by pH and temperature, with higher values of each shifting the equilibrium toward free ammonia, thus increasing the risk of inhibition.

To manage ammonia effectively in ASB1, key parameters—moisture content, pH, and temperature—are carefully regulated. The low-moisture conditions in ASB1 favor dry, lignocellulosic feedstocks, which naturally contain less nitrogen than wetter, nitrogen-rich materials. As a result, nitrogen input is limited, reducing potential ammonia concentrations. Additionally, maintaining a pH range of 6.5 to 8.0 helps keep most ammonia in the less toxic ammonium ion form rather than as free ammonia. This pH range, combined with a mesophilic temperature range, minimizes the conversion of ammonium to free ammonia, further reducing toxicity.

### ASB1 Main Function

In one example, the main function of ASB1 is to facilitate enzymatic hydrolysis, acetogenesis, and partial acidogenesis of lignocellulosic feedstock, rather than extensive biogas production. While a limited amount of methane may be generated, the majority of biogas production is reserved for a downstream processing stage specifically optimized for methanogenesis.

### PROCESSING STAGE 2 ASB2 Thermophilic Degradation Technologies

In one example, the disclosed biogas production technologies utilize a second processing stage (ASB2) that operates under thermophilic conditions, typically between 65° C. and 85° C., with a pH maintained between 6.5 and 8.0. This stage further processes the effluent derived from ASB1 to break down residual lignocellulosic material and microbial cell walls to produce additional methanogenic precursors. This effluent, enriched with soluble intermediates and microbial material, enters ASB2's microbial environment specifically optimized for high-temperature processing. Thermophilic conditions enhance enzymatic stability and activity, accelerating the breakdown of recalcitrant substrates and reducing contamination by mesophilic organisms and pathogens, thus promoting a synthetic microbial community suited for continued degradation of complex organic matter.

In one example, the synthetic microbial community in ASB2 includes thermophilic organisms selected from Caldicellulosiruptor spp., Clostridium thermocellum, Thermoanaerobacterium saccharolyticum, Thermobifida fusca, Thermomonospora curvata, Thermomyces lanuginosus, and/or Myceliophthora thermophila, which are capable of effectively hydrolyzing cellulose, hemicellulose (including xylan and mannan), and, to some extent, lignin at elevated temperatures. These microbes produce a diverse array of exozymes—including cellulases, xylanases, mannanases, chitinases, proteases, and auxiliary lignin-degrading enzymes—that work synergistically to deconstruct residual plant cell wall components and microbial cell walls from ASB1 into simpler compounds.

This enzymatic breakdown releases a variety of soluble sugars, volatile fatty acids (VFAs), amino acids, and other intermediary metabolites, which remain primarily in their anionic forms under the slightly alkaline to neutral conditions maintained in ASB2. Key VFAs, such as acetate, propionate, and butyrate, along with other breakdown products like alcohols and amino acid derivatives, generated in this process, serve as ideal precursors for methanogenesis in a downstream stage of anaerobic digestion, thereby enhancing the overall biogas production potential.

### Example Stage 2 Anaerobic Secretome Bioreactor

In one example, a second-stage anaerobic secretome bioreactor, such as ASB2 shown in FIG. 2, includes a digester vessel, such as vessel 22, and is configured to break down feedstock or other substrate, such as feedstock 24. The vessel includes space, such as space 26, to allow for the accumulation and removal of gases produced during substrate digestion. Vessel 22 may be equipped with a thermally insulated chamber to maintain the elevated temperatures required for efficient thermophilic degradation. A temperature control system ensures uniform temperatures throughout the vessel, supporting consistent enzyme activity and stable microbial function. This setup is optimized for processing high-moisture substrates to facilitate the thorough breakdown of complex organic compounds under thermophilic, anaerobic conditions.

Vessel 22 may include multiple ports to facilitate various stages of the anaerobic digestion process. An inlet port, such as port 23, is configured for the controlled introduction of ASB1 effluent into ASB2, providing the primary substrate for thermophilic processing. A discharge port, such as port 25, allows for the removal of the enriched liquid effluent produced in ASB2. This effluent, rich in methanogenic precursors, serves as a high-value input for a subsequent methanogenesis stage of processing. A gas release port, such as port 27, vents gaseous byproducts generated during thermophilic digestion, preventing pressure buildup and ensuring safe, stable operation within the bioreactor. Additional ports, such as port 28, may be included for similar or alternative functions as needed, with positioning and configuration tailored to optimize ASB2's performance and accessibility.

ASB2 may include an internal stirring or mixing mechanism(s), such as mechanism 20, to ensure uniform distribution of the substrate, enhancing contact between the microbial community and substrate within the bioreactor. For example, a mechanical agitator or impeller system may be used to maintain the substrate in a consistent slurry, ensuring even dispersion of solid and liquid phases throughout the bioreactor. Additional or alternative mixing mechanisms may also be employed as needed. These mixing capabilities facilitate uniform hydrolysis, accelerating the degradation of residual plant cell wall components and microbial cell walls.

ASB2 may further benefit from a pH monitoring and control system(s), such as system 21, to maintain an optimal pH range conducive to thermophilic microbial activity and enzyme function. These systems can include pH sensors strategically positioned within the vessel to provide continuous, real-time measurements of the substrate environment. When pH fluctuations are detected, buffering agents—such as bicarbonate or other suitable alkaline or acidic additives—can be automatically introduced through a controlled dosing mechanism. In some cases, effluent from another stage, such as a methanogenic digestion stage of processing, may be repurposed as a buffering agent, enhancing both process efficiency and sustainability. This pH control capability helps maintain a stable reactor environment, typically within a pH range of 6.5 to 8.0, preventing acidification that could inhibit microbial activity and enzyme function.

Vessel 22 may be oriented in various positions, such as vertical, horizontal, or angled, based on the specific design requirements and operational goals of the anaerobic digestion process. Orientation affects factors such as feedstock flow dynamics, mixing efficiency, gas-liquid separation, and ease of feeding and discharge. For example, a vertical orientation is often advantageous for wet reactors, promoting natural stratification of gas, liquid, and solid phases. The selected orientation should facilitate optimal retention times, degradation efficiency, and convenient feeding, discharge, operation, maintenance, and monitoring.

### ASB2 Characteristics

In one example, ASB2 is configured to create and maintain specific internal conditions essential for efficient thermophilic anaerobic metabolism. Three key parameters—moisture level, pH, and temperature—are managed to support the optimal activity of the synthetic microbial community responsible for further breaking down ASB1 effluent. Each of these parameters is critical to maintaining a stable and balanced microbial environment within ASB2. Moisture levels are carefully regulated to support microbial activity while conserving energy, with a high-moisture content tailored to the needs of a vertical wet thermophilic anaerobic system. The vertical orientation facilitates efficient mixing, natural stratification of gas, liquid, and solid phases, and enhances microbial contact with the substrate. pH is maintained within a range that minimizes microbial inhibition, stabilizes enzyme function, and fosters optimal conditions for substrate degradation. Additionally, a thermophilic temperature range is controlled to balance microbial activity with energy efficiency, promoting steady substrate degradation.

### ASB2 Moisture Levels and Sources

In one example, ASB2, a vertical wet reactor, operates with moisture levels suitable for high-moisture anaerobic metabolism, creating an ideal environment for breaking down residual plant cell wall components and microbial cell walls. Water and/or other fluids may be introduced into the vessel to achieve optimal moisture levels. In one example, fluid is added through a port in vessel 22, such as port 28.

Alternatively, or additionally, effluent from a prior or subsequent processing stage may be introduced into the vessel to enhance both moisture content and microbial activity. This effluent, particularly if rich in bicarbonate, helps maintain optimal pH levels and provides essential buffering capacity to support stable microbial growth and enzymatic activity. Recycling liquid effluent, which may carry active microorganisms and beneficial nutrients, improves overall process efficiency, fostering a well-balanced microbial community that enhances substrate breakdown and biogas production.

Alternatively, or additionally, a substrate or substrate supplement such as waste-activated sludge (WAS) may be introduced to the vessel to enhance microbial activity by supplying additional nutrients. This supplementation supports a balanced microbial community, fostering optimal conditions for microbial colonization and efficient substrate breakdown.

### ASB2 pH Management and Stability

In one example, to optimize soluble intermediate production, ASB2 is maintained within an optimal pH range of approximately 6.5 to 8.0. This range supports the metabolic activities of the synthetic microbial community that contributes to breaking down residual plant cell wall components and microbial cell walls into simpler compounds. Maintaining this pH range promotes a balanced microbial community, enabling each microbial group to efficiently perform its role in substrate degradation, thereby enhancing production of soluble compounds.

To control pH, buffering agents like bicarbonate may be introduced, either through liquid effluents or direct supplementation, to counteract potential pH drops. Regular pH monitoring allows for timely adjustments, ensuring the microbial community remains balanced and resilient. Maintaining pH within this optimal range creates an environment conducive to efficient substrate degradation ASB2.

### ASB2 Temperature Management

In one example, ASB2 operates within a controlled thermophilic temperature range, typically maintained between approximately 65° C. and 85° C. This temperature range optimally supports the metabolic activities of the synthetic microbial community involved in this second stage of processing. Thermophilic conditions enable the microbial community to thrive and work synergistically. Maintaining this temperature range enhances enzyme production and promotes the breakdown of residual plant cell wall components and microbial cell walls into simpler compounds. Most of these compounds are further converted by methanogens into biogas in a downstream processing stage.

Thermophilic temperatures also enhance system resilience by supporting a diverse microbial community capable of degrading residual plant cell wall components and microbial cell walls under high-moisture conditions. This stability enables ASB2 to achieve consistent degradation yields and maintain a robust microbial community that can adapt to variations in substrate composition. By maintaining temperatures within this thermophilic range, ASB2 promotes optimal microbial performance, ensuring efficient substrate conversion and steady substrate degradation throughout the bioreactor.

### ASB2 Substrate Breakdown

In one example, ASB2 is configured to maximize substrate exposure to the synthetic microbial community, promoting efficient enzymatic hydrolysis under high-moisture, thermophilic conditions, typically maintained at a temperature range of approximately 65° C. to 85° C. and a pH between 6.5 and 8.0. The stable and synergistic microbial community adapts effectively to these conditions, producing a variety of exozymes—both secreted and cell-bound, such as cellulases, hemicellulases, and proteases—that collaboratively break down residual microbial cell walls and other organic compounds from the ASB1 effluent. This process transforms complex polysaccharides, proteins, and other macromolecules into simpler, soluble compounds. Following enzymatic hydrolysis, a limited portion of these simpler compounds may be metabolized by the microbial community within ASB2, while the majority of soluble compounds accumulate in the effluent for downstream methanogenic processing.

As the effluent exits ASB2, it primarily comprises a nutrient-rich mix of partially degraded organic compounds, including volatile fatty acid anions, residual simple sugars, and other soluble byproducts. This effluent serves as a methanogenesis-ready substrate, optimized for subsequent methanogenic digestion and biogas production. In some examples, the lignin and unconverted fibers are removed from the effluent and may be repurposed as soil amendments, biochar, or even fuel, depending on the desired end use and process economics.

### ASB2 Carbon and Nitrogen Management

In one example, maintaining a balanced carbon-to-nitrogen (C/N) ratio in ASB2 supports stable microbial activity under thermophilic conditions, helping to control the production of inhibitory compounds, such as ammonia and organic acids. Although the lignocellulosic feedstock in ASB1 may contribute minimal nitrogen, a C/N ratio between 20:1 and 30:1 in ASB2 ensures sufficient nitrogen availability for microbial growth without leading to excess ammonia production. This balanced ratio also prevents an overabundance of carbon that could result in organic acid buildup, promoting optimal microbial function and efficient substrate degradation.

Additionally, maintaining a pH between 6.5 and 8.0 further supports microbial stability by ensuring that organic acids remain primarily in their less inhibitory, anionic forms. This pH range reduces the toxicity risk from organic acids, fostering syntrophic relationships among microbial species that drive efficient substrate breakdown.

### ASB2 Ammonia Management

In one example, ammonia can act as an inhibitor in ASB2. Although the effluent from ASB1 may contain minimal nitrogen, any ammonia produced during the breakdown of residual nitrogen compounds could accumulate in ASB2's high-moisture environment. Such ammonia typically exists in two forms: ammonium ions (NH₄⁺) and free ammonia (NH₃), with free ammonia being more toxic due to its ability to diffuse across microbial cell membranes, disrupting cellular functions. The balance between these forms is influenced by pH and temperature, with higher values of each shifting the equilibrium toward free ammonia, thus increasing the risk of inhibition.

To manage ammonia effectively in ASB2, key parameters—moisture content, pH, and temperature—are carefully regulated. The high-moisture conditions in ASB2 are optimized for thermophilic microbial activity, while also contributing to an environment that dilutes nitrogen levels, naturally reducing potential ammonia concentrations. Maintaining a pH between 6.5 and 8.0 helps keep ammonia primarily in the less toxic ammonium ion form rather than free ammonia. This pH range, coupled with the thermophilic temperature range, minimizes the conversion of ammonium to free ammonia, further reducing the risk of toxicity. By controlling these conditions, ASB2 supports stable microbial performance and efficient substrate degradation.

### ASB2 Main Function

In one example, the main function of ASB2 is to pasteurize and further break down the effluent from ASB1 through enzymatic hydrolysis, acetogenesis, and acidogenesis, creating an ideal substrate for final methanogenesis. While ASB2 may produce a large amount of carbon dioxide, its primary objective is to generate an effluent optimized for downstream methanogenic processing. This optimization is aimed at maximizing biogas quality, with minimal carbon dioxide content, in the final biogas product.

### THREE-STAGE SYSTEM

### Methanogenic Processing Technologies

FIG. 3 illustrates an example three-stage system, system 300, that is configured for optimized lignocellulosic feedstock digestion and that comprises ASB1, ASB2, and methanogenic digester 3 (MD3). In general, effluent is transferred from ASB1 via any suitable means, such as means 312, into ASB2, effluent is transferred from ASB2 via any suitable means, such as means 323, into MD3 via at least one port, such as port 33, and system 300 is controlled via any suitable control system(s), such as control system 30. Such control typically provides comprehensive monitoring and management of all aspects of system 300's operation. Control system 30 may optionally include system 11 and/or system 21.

In one example, a third-stage methanogenic digester, such as MD3 as illustrated in FIG. 3, includes a digester vessel, such as vessel 32, and is configured to convert substrate, such as substrate 34, into primarily methane. The vessel includes space, such as space 36, to allow for the accumulation of methane and other gases produced during methanogenic digestion, which gases can be collected via at least one port, such as port 38. Vessel 32 also includes at least one discharge port, such as port 35, for discharging liquid and/or solid effluents. Additional ports suitable for similar or alternative purposes may be included for similar or alternative functions as needed, with positioning and configuration tailored to optimize MD3 performance and accessibility.

MD3 may include an internal stirring or mixing mechanism(s), such as mechanism 30, to ensure uniform distribution of the substrate, enhancing contact between the methanogenic microbial community and substrate within the bioreactor. For example, a mechanical agitator or impeller system may be used to maintain the substrate in a consistent slurry, ensuring even dispersion of solid and liquid phases throughout the bioreactor. Additional or alternative mixing mechanisms may also be employed as needed.

MD3 may further benefit from a pH monitoring and control system, similar to system 21, to maintain an optimal pH range conducive to methanogenic microbial activity. This system can include strategically placed pH sensors within the vessel to provide continuous, real-time measurements of the substrate environment. When pH fluctuations are detected, buffering agents-such as bicarbonate or other suitable alkaline or acidic additives- can be introduced through a controlled dosing mechanism. In some cases, effluent from another stage may be repurposed as a buffering agent, enhancing both process efficiency and sustainability. This pH control capability stabilizes the reactor environment, typically within a pH range of 6.8 to 8.0, preventing acidification or alkalization that could inhibit methanogenic activity and biogas production.

### MD3 Methanogenic Microbial Community

In one example, the microbial community in MD3 is specifically adapted to optimize methane production through methanogenesis. This methanogenic microbial community is dominated by methanogenic archaea, including acetoclastic methanogens such as Methanosaeta and Methanosarcina, which convert acetate into methane and carbon dioxide, hydrogenotrophic methanogens such as Methanobacterium and Methanobrevibacter, which utilize hydrogen and carbon dioxide to produce methane, and Methanosarcinales, Methanobacteriales and Methanomassiliicoccales that metabolize methylated compounds.

Syntrophic bacteria, such as Methanobacterium and Methanobrevibacter, play a crucial supporting role by oxidizing volatile fatty acids (e.g., propionate and butyrate) and alcohols into methanogenic precursors like acetate, hydrogen, and carbon dioxide. This oxidation process is thermodynamically favorable only under low hydrogen partial pressures, maintained by the activity of hydrogenotrophic methanogens. Hydrolytic and fermentative bacteria, including genera such as Clostridium and Prevotella, further contribute by breaking down residual proteins, lipids, and carbohydrates into simpler substrates, ensuring a steady supply of precursors for methanogenesis.

This highly specialized and collaborative microbial ecosystem thrives under anaerobic conditions, neutral to slightly alkaline pH (6.8-8.0), and either mesophilic (35-40° C.) or thermophilic (50-60° C.) temperature ranges, depending on operational design. Together, these microorganisms efficiently convert the methanogenesis-ready substrate from ASB2 into high-yield biogas with a methane content typically ranging from 60% to 65%, maximizing energy recovery and enhancing the sustainability of the system.

### MD3 Main Function

In one example, the main function of MD3 is to facilitate methanogenesis, the final stage of anaerobic digestion, where methanogenic microorganisms convert volatile fatty acids and other intermediates into biogas, primarily methane. MD3 operates under controlled conditions to optimize methane production while minimizing carbon dioxide output, producing high-quality biogas. Any residual compounds from prior stages are efficiently converted, ensuring maximal bioconversion efficiency in this methanogenic stage.

As a secondary function, MD3 also produces bicarbonate in its effluent as a byproduct of methanogenesis. This bicarbonate contributes to the reactor's buffering capacity and helps maintain a stable pH, and it can be repurposed to buffer earlier stages of processing, enhancing overall process stability and efficiency.

System 300 employs transfer means, such as means 331 and 332, and flow control means, such as valves 301 and 302, to recycle MD3's bicarbonate-rich effluent into ASB1 and/or ASB2 as needed.

### First Example of the Three-Stage System

In one example, the three-stage system processes high-lignin feedstock, achieving distinct carbon conversion and biogas characteristics at each stage. During the first stage (ASB1), approximately 30% of the carbon in the feedstock is converted into biogas containing about 55% carbon dioxide (CO₂). This occurs under mesophilic, low-moisture conditions. ASB1 facilitates initial enzymatic hydrolysis, partial acidogenesis, and acetogenesis of the lignocellulosic material, breaking it down into intermediate products.

The second stage (ASB2) operates under thermophilic, high-moisture conditions, producing significant amounts of carbon dioxide but little to no methane. These conditions promote the further hydrolysis of residual lignocellulose, microbial cell walls, and organic intermediates. These conditions do not support methanogenesis, as methanogenic microorganisms are typically not active under the hydrolytic and thermophilic environment of ASB2. The resulting methanogenic precursors are optimized for conversion in the subsequent methanogenesis stage.

In the third stage (MD3), approximately 15-30% of the carbon from the original feedstock is converted into biogas containing 60-65% methane. The methanogenic digester efficiently converts the precursors generated in earlier stages into methane while minimizing carbon dioxide production. This multi-stage process maximizes biogas yield and ensures a high-quality product.

The multi-stage system's ability to convert approximately 45-60% of the original feedstock carbon into biogas represents a significant advancement in the anaerobic digestion industry, particularly for high-lignin materials. Conventional single-stage digesters typically achieve much lower conversion rates for lignin-rich feedstocks, often below 30%, due to the recalcitrant nature of lignin. By leveraging sequential optimization across the hydrolysis, precursor generation, and methanogenesis stages, this system achieves biogas yields that rival or exceed those of advanced multi-stage systems while expanding the range of economically viable feedstocks. These results underscore the system's potential to maximize renewable energy generation and enhance the sustainability of waste management practices.

When high-energy supplemental feedstocks such as waste-activated sludge (WAS), fats, oils, and grease (FOG), brewery bottoms, or manure are introduced, the overall biogas yield can significantly exceed the baseline of 45-60% conversion achieved with high-lignin feedstocks alone. These supplemental materials provide readily digestible substrates, including lipids, proteins, and sugars, that enhance methanogenesis in MD3 and accelerate microbial activity across all stages.

The addition of these materials can increase overall carbon conversion efficiency by 10-20%, resulting in total system conversion rates of 55-80%, depending on the feedstock blend and operational conditions such as retention time, temperature, and pH. For example, FOG contributes energy-dense lipids that significantly enhance methane production. WAS, on the other hand, provides microbial biomass and nutrients that stimulate microbial activity and improve overall system performance.

This capability to integrate supplemental feedstocks enhances the system's versatility. It allows operators to maximize renewable energy recovery from diverse waste streams while maintaining robust and adaptable operation.

### Second Example of the Three-Stage System

In another example, ASB1 not only facilitates the initial hydrolysis of high-lignin feedstock but also serves as a bioreactor for cultivating microbial biomass. Under the mesophilic, low-moisture conditions of ASB1, lignocellulose-degrading fungi from the genus Neocallimastigomycota and associated microbial consortia thrive, proliferating as they metabolize the feedstock. The microbial biomass produced in ASB1, consisting of fungal hyphae, bacterial cells, and microbial exudates, represents a valuable intermediate substrate. These exudates contribute to feedstock breakdown and act as precursors for subsequent digestion stages.

A portion of this microbial biomass is transferred with the partially degraded feedstock to ASB2, where thermophilic conditions further hydrolyze the microbial cells. This process releases intracellular enzymes, organic compounds, and additional carbon substrates, including volatile fatty acids (VFAs) and/or their anions, which are critical intermediates for methanogenesis in MD3. The increased availability of these precursors, along with the microbial residues, in turn supports higher methane yields in MD3, improving overall energy recovery efficiency. Notably, the microbial biomass cultivated in ASB1 is expected to account for approximately one-half of the total methane produced by the system, underscoring its critical role in biogas generation.

The deliberate cultivation of microbial biomass in ASB1 offers several advantages. First, it accelerates the degradation of lignocellulosic material by ensuring a high concentration of active enzymes and microbial consortia in the system. Second, the hydrolysis of microbial biomass in ASB2 provides additional readily degradable substrates, improving the efficiency and productivity of downstream methanogenesis. Third, the process increases system robustness by maintaining a self-sustaining microbial population that adapts to the feedstock composition over time.

The synergistic use of ASB1 as both a hydrolysis reactor and a microbial biomass cultivation system enables more efficient carbon utilization across the entire process. By designing ASB1 to maximize microbial growth, the system leverages the dual role of microorganisms as both agents of lignocellulose breakdown and substrates for further digestion. This approach not only enhances methane yields in MD3 but also improves the overall sustainability and cost-effectiveness of the system. Optimizing ASB1 microbial growth ensures a steady supply of high-quality substrates for ASB2 and MD3, enhancing system-wide performance. The significant contribution of ASB1-grown biomass to total methane production demonstrates the importance of optimizing its operation for system-wide performance.

### Other Examples of the Three-Stage System

In other examples, including combinations of the first and second examples above, the specific outputs of each stage may vary depending on feedstock composition, system conditions, or operational adjustments. For instance, incorporating low-lignin materials such as food waste may reduce hydrolysis times and increase overall methane yields. Adjustments to retention times, temperature ranges, moisture levels, pH, or other factors including but not limited to agitation rates or microbial inoculation strategies can further optimize the process for specific feedstock compositions and system performance.

### Conclusion

In a first example, a method of breaking down lignocellulosic substrate in an anaerobic secretome bioreactor comprising inoculating the anaerobic secretome bioreactor with microbiota derived from at least one large herbivore, the inoculating resulting in a microbiome within the anaerobic secretome bioreactor that comprises at least one species from the genus Neocallimastigomycota and symbiotic species of bacteria and archaea that are capable of producing a secretome of exozymes capable of hydrolyzing and solubilizing a substantial portion of the lignocellulosic substrate; maintaining, in the inoculated anaerobic secretome bioreactor, the lignocellulosic substrate and the microbiome at a temperature substantially within a mesophilic temperature range; hydrolyzing and solubilizing, by the secretome of exozymes in the inoculated anaerobic secretome bioreactor, the substantial portion of the lignocellulosic substrate, resulting in soluble compounds; and metabolizing, by the microbiome in the inoculated anaerobic secretome bioreactor, a portion of the hydrolyzed and solubilized lignocellulosic substrate, resulting in sufficient cellular Gibbs energy to support growth of the microbiome.

In the first example, the anaerobic secretome bioreactor is configured as a horizontal dry bioreactor; further comprising maintaining the lignocellulosic substrate and the microbiome in the inoculated anaerobic secretome bioreactor at a water volume percentage of substantially 1 to 15; further comprising producing, by the inoculated anaerobic secretome bioreactor, effluent that comprises microbial biomass from the microbiome; wherein the effluent further comprises a portion of the soluble compounds; further comprising producing, by the secretome of exozymes and the microbiome in the inoculated anaerobic secretome bioreactor, biogas; further comprising maintaining, in the inoculated anaerobic secretome bioreactor, the lignocellulosic substrate and the microbiome at a pH substantially within a pH range of 6.5 and 8.0.

In a second example, an anaerobic secretome bioreactor comprising a digester vessel is configured for being inoculated with microbiota derived from at least one large herbivore, the inoculating resulting in a microbiome within the digester vessel that comprises at least one species from the genus Neocallimastigomycota and symbiotic species of bacteria and archaea that are capable of producing a secretome of exozymes capable of hydrolyzing and solubilizing a substantial portion of the lignocellulosic substrate; the anaerobic secretome bioreactor configured for maintaining, in the inoculated digester vessel, the lignocellulosic substrate and the microbiome at a temperature substantially within a mesophilic temperature range, wherein the secretome of exozymes is capable of hydrolyzing and solubilizing, in the inoculated digester vessel, a substantial portion of the lignocellulosic substrate, resulting in soluble compounds, and wherein the microbiome is capable of metabolizing, in the inoculated digester vessel, a portion of the hydrolyzed and solubilized lignocellulosic substrate, resulting in sufficient cellular Gibbs energy to support growth of the microbiome.

In the second example, the anaerobic secretome bioreactor is configured as a horizontal dry bioreactor; further configured for maintaining the lignocellulosic substrate and the microbiome in the inoculated digester vessel at a water volume percentage of substantially 1 to 15; further configured for producing effluent that comprises microbial biomass from the microbiome; wherein the effluent further comprises a portion of the soluble compounds; further configured for producing, by the secretome of exozymes and the microbiome in the inoculated digester vessel, biogas; further configured for maintaining, in the inoculated digester vessel, the lignocellulosic substrate and the microbiome at a pH substantially within a pH range of 6.5 and 8.0.

In a third example, a method of breaking down lignocellulosic substrate in a system comprising inoculating a first anaerobic secretome bioreactor of the system with microbiota derived from at least one large herbivore, the inoculating resulting in a microbiome within the first anaerobic secretome bioreactor that comprises at least one species from the genus Neocallimastigomycota and symbiotic species of bacteria and archaea that are capable of producing a secretome of exozymes capable of hydrolyzing and solubilizing a substantial portion of the lignocellulosic substrate; maintaining, by the system, the lignocellulosic substrate and the microbiome in the inoculated first anaerobic secretome bioreactor at a temperature substantially within a mesophilic temperature range; first breaking down, by the secretome of exozymes and the microbiome in the inoculated first anaerobic secretome bioreactor, the substantial portion of the lignocellulosic substrate resulting in biogas and first effluent that comprises microbial biomass from the microbiome, partially degraded organic compounds, and first soluble compounds; communicating the first effluent from the inoculated first anaerobic secretome bioreactor to a second anaerobic secretome bioreactor of the system that comprises a synthetic microbial community comprising at least one type of microorganism selected from thermophilic anaerobic microorganisms capable of acting as acidogens and acetogens and of producing a secretome of exozymes capable of hydrolyzing and solubilizing a substantial portion of the first effluent; second breaking down, by the secretome of exozymes and the synthetic microbial community in the second anaerobic secretome bioreactor, the substantial portion of the first effluent resulting in a methanogenesis-ready substrate; communicating the methanogenesis-ready substrate from the second anaerobic secretome bioreactor to a methanogenic digester of the system comprising a methanogenic microbial community capable of producing methane from the methanogenesis-ready substrate; and converting, by the methanogenic microbial community in the methanogenic digester, a substantial portion of the methanogenesis-ready substrate to methane.

In the third example, further comprising maintaining, by the system, the first effluent and the synthetic microbial community in the second anaerobic secretome bioreactor at a temperature substantially within a thermophilic temperature range; further comprising maintaining, by the system, the first effluent and the synthetic microbial community in the second anaerobic secretome bioreactor at a pH substantially within a pH range of 6.5 and 8.0; wherein the at least one type of microorganism selected from thermophilic anaerobic microorganisms includes Caldicellulosiruptor spp., Clostridium thermocellum, Thermoanaerobacterium saccharolyticum, Thermobifida fusca, Thermomonospora curvata, Thermomyces lanuginosus, and/or Myceliophthora thermophila; further comprising maintaining, by the system, the methanogenesis-ready substrate and the methanogenic microbial community in the methanogenic digester at a temperature substantially within a thermophilic temperature range; further comprising maintaining, by the system, the methanogenesis-ready substrate and the methanogenic microbial community in the methanogenic digester at a pH substantially within a pH range of 6.8 and 8.0; wherein the methanogenic microbial community includes Methanosaeta, Methanosarcina, Methanobacterium, Methanobrevibacter, Methanobacterium, Methanobrevibacter, Clostridium, Prevotella, Methanosarcinales, Methanobacteriales, and/or Methanomassiliicoccales.

1. A method of breaking down lignocellulosic substrate in an anaerobic secretome bioreactor, the method comprising:
inoculating the anaerobic secretome bioreactor with microbiota derived from at least one large herbivore, the inoculating resulting in a microbiome within the anaerobic secretome bioreactor that comprises at least one species from the genus Neocallimastigomycota and symbiotic species of bacteria and archea that are capable of producing a secretome of exozymes capable of hydrolyzing and solubilizing a substantial portion of the lignocellulosic substrate;
maintaining, in the inoculated anaerobic secretome bioreactor, the lignocellulosic substrate and the microbiome at a temperature substantially within a mesophilic temperature range;
hydrolyzing and solubilizing, by the secretome of exozymes in the inoculated anaerobic secretome bioreactor, the substantial portion of the lignocellulosic substrate, resulting in soluble compounds; and
metabolizing, by the microbiome in the inoculated anaerobic secretome bioreactor, a portion of the hydrolyzed and solubilized lignocellulosic substrate, resulting in sufficient cellular Gibbs energy to support growth of the microbiome.

2. The method of claim 1, wherein the anaerobic secretome bioreactor is configured as a horizontal dry bioreactor.

3. The method of claim 1, further comprising maintaining the lignocellulosic substrate and the microbiome in the inoculated anaerobic secretome bioreactor at a water volume percentage of substantially 1 to 15.

4. The method of claim 1, further comprising producing, by the inoculated anaerobic secretome bioreactor, effluent that comprises microbial biomass from the microbiome.

5. The method of claim 4, wherein the effluent further comprises a portion of the soluble compounds.

6. The method of claim 1, further comprising producing, by the secretome of exozymes and the microbiome in the inoculated anaerobic secretome bioreactor, biogas.

7. The method of claim 1, further comprising maintaining, in the inoculated anaerobic secretome bioreactor, the lignocellulosic substrate and the microbiome at a pH substantially within a pH range of 6.5 and 8.0.

8. An anaerobic secretome bioreactor comprising:
a digester vessel configured for being inoculated with microbiota derived from at least one large herbivore, the inoculating resulting in a microbiome within the digester vessel that comprises at least one species from the genus Neocallimastigomycota and symbiotic species of bacteria and archea that are capable of producing a secretome of exozymes capable of hydrolyzing and solubilizing a substantial portion of the lignocellulosic substrate;
the anaerobic secretome bioreactor configured for maintaining, in the inoculated digester vessel, the lignocellulosic substrate and the microbiome at a temperature substantially within a mesophilic temperature range, wherein the secretome of exozymes is capable of hydrolyzing and solubilizing, in the inoculated digester vessel, a substantial portion of the lignocellulosic substrate, resulting in soluble compounds, and wherein the microbiome is capable of metabolizing, in the inoculated digester vessel, a portion of the hydrolyzed and solubilized lignocellulosic substrate, resulting in sufficient cellular Gibbs energy to support growth of the microbiome.

9. The anaerobic secretome bioreactor of claim 8, wherein the anaerobic secretome bioreactor is configured as a horizontal dry bioreactor.

10. The anaerobic secretome bioreactor of claim 8, further configured for maintaining the lignocellulosic substrate and the microbiome in the inoculated digester vessel at a water volume percentage of substantially 1 to 15.

11. The anaerobic secretome bioreactor of claim 8, further configured for producing effluent that comprises microbial biomass from the microbiome.

12. The anaerobic secretome bioreactor of claim 11, wherein the effluent further comprises a portion of the soluble compounds.

13. The anaerobic secretome bioreactor of claim 8, further configured for producing, by the secretome of exozymes and the microbiome in the inoculated digester vessel, biogas.

14. The anaerobic secretome bioreactor of claim 8, further configured for maintaining, in the inoculated digester vessel, the lignocellulosic substrate and the microbiome at a pH substantially within a pH range of 6.5 and 8.0.

15. A method of breaking down lignocellulosic substrate in a system, the method comprising:
inoculating a first anaerobic secretome bioreactor of the system with microbiota derived from at least one large herbivore, the inoculating resulting in a microbiome within the first anaerobic secretome bioreactor that comprises at least one species from the genus Neocallimastigomycota and symbiotic species of bacteria and archea that are capable of producing a secretome of exozymes capable of hydrolyzing and solubilizing a substantial portion of the lignocellulosic substrate;
maintaining, by the system, the lignocellulosic substrate and the microbiome in the inoculated first anaerobic secretome bioreactor at a temperature substantially within a mesophilic temperature range;
first breaking down, by the secretome of exozymes and the microbiome in the inoculated first anaerobic secretome bioreactor, the substantial portion of the lignocellulosic substrate resulting in biogas and first effluent that comprises microbial biomass from the microbiome, partially degraded organic compounds, and first soluble compounds;
communicating the first effluent from the inoculated first anaerobic secretome bioreactor to a second anaerobic secretome bioreactor of the system that comprises a synthetic microbial community comprising at least one type of microorganism selected from thermophilic anaerobic microorganisms capable of acting as acidogens and acetogens and of producing a secretome of exozymes capable of hydrolyzing and solubilizing a substantial portion of the first effluent;
second breaking down, by the secretome of exozymes and the synthetic microbial community in the second anaerobic secretome bioreactor, the substantial portion of the first effluent resulting in a methanogenesis-ready substrate;
communicating the methanogenesis-ready substrate from the second anaerobic secretome bioreactor to a methanogenic digester of the system comprising a methanogenic microbial community capable of producing methane from the methanogenesis-ready substrate; and
converting, by the methanogenic microbial community in the methanogenic digester, a substantial portion of the methanogenesis-ready substrate to methane.

16. The method of claim 15, further comprising:
maintaining, by the system, the first effluent and the synthetic microbial community in the second anaerobic secretome bioreactor at a temperature substantially within a thermophilic temperature range; and
maintaining, by the system, the first effluent and the synthetic microbial community second anaerobic secretome bioreactor at a pH substantially within a pH range of 6.5 and 8.0.

17. The method of claim 15, wherein the at least one type of microorganism selected from thermophilic anaerobic microorganisms includes Caldicellulosiruptor spp., Clostridium thermocellum, Thermoanaerobacterium saccharolyticum, Thermobifida fusca, Thermomonospora curvata, Thermomyces lanuginosus, and/or Myceliophthora thermophila.

18. The method of claim 15, further comprising:
maintaining, by the system, the methanogenesis-ready substrate and the methanogenic microbial community in the methanogenic digester at a temperature substantially within a thermophilic temperature range.

19. The method of claim 15, further comprising:
maintaining, by the system, the methanogenesis-ready substrate and the methanogenic microbial community in the methanogenic digester at a pH substantially within a pH range of 6.8 and 8.0.

20. The method of claim 15, wherein the methanogenic microbial community includes Methanosaeta, Methanosarcina, Methanobacterium, Methanobrevibacter, Methanobacterium, Methanobrevibacter, Clostridium, Prevotella, Methanosarcinales, Methanobacteriales, and/or Methanomassiliicoccales.
